(19) United States
(12) Patent Application Publication
(10) Pub. No.: US 2008/0292684 A1
(43) Pub. Date: Nov. 27, 2008

(54) Device for Delivery of Active Principles

(75) Inventors: Paolo Colombo, Parma (IT); Patrizia Santi, Salsomaggiore (IT); Sara Nicoli, Parma (IT); Cristina Padula, Trivigno (IT); Fabio Marra, Monteroni di Lecce (IT)

Correspondence Address: OHLANDT, GREELEY, RUGGIERO & PERLE, LLP, ONE LANDMARK SQUARE, 10TH FLOOR, STAMFORD, CT, 06901, US

(73) Assignee: Laboratorio Italiano Biochimico Farmaceutico Lisapharma S.P.A

(21) Appl. No.: 11/658,282

(22) PCT Filed: Jul. 22, 2005
(86) PCT No.: PCT/EP2005/053581

§ 371 Date: Jan. 23, 2007

(30) Foreign Application Priority Data
Jul. 23, 2004 (IT) MI2004A001492

(51) Int. Cl. A61F 13/00

(52) U.S. Cl. 424/449

(57) ABSTRACT

A device for the delivery of active principles for dermal and, in particular, transdermal application having the form of a two-layered patch consisting of a first layer having a homogeneous composition and comprising at least one active principle, a water-soluble film-forming agent and a hydrophilic adhesive polymer, and of a second layer joined in a permanent manner to the first and having a water vapour permeability of less than 500 g/m² in 24 hours.

### BACKGROUND

1. Field of the Invention

The present disclosure relates to a device for the delivery of active principles for dermal and, in particular, transdermal application.

2. Discussion of the Background of the Art

The administration of drugs through the skin, known as transdermal administration, has undergone a considerable boost in recent years, by virtue of the development of new systems for delivering a substance to the skin, where the term system means a device formed of various individual elements which jointly contribute to the functioning of the system itself. These delivery systems or devices for the skin are known as dermal or transdermal patches, being composed of an adhesive, supported by a backing which is designed to maintain the drug in contact with the skin.

A typical patch for dermal or transdermal application consists of various elements which are formed in layers of various materials, placed one over the other. Said layers, superimposed in the following sequence, can be typically:

1. a backing of various materials, which is a transparent or opaque flexible film. It acts as the supporting structure of the patch itself and gives consistency to the patch, allowing it to be positioned and maintained in contact with the skin;
2. a deposit of solid, semisolid or liquid active principle, containing the active substance;
3. a membrane interposed between the active principle deposit and the skin, with the task of controlling active substance release rate. This element is not always present;
4. an adhesive which ensures contact between the patch and the skin surface. Said adhesive must be permeable to the active principle.

In general, all transdermal patches are packaged in foil sachets, the adhesive being covered by a sheet of material (release liner), coated with silicone polymers or fluoropolymers, to avoid unwanted adhesions during patch handling and storage. This element is not involved in the therapeutic application of the patch and is removed before applying to the skin.

From an application viewpoint, transdermal patches are a very practical pharmaceutical form relative to traditional forms of administration. Indeed, the transdermal administration method has many advantages compared to conventional means, such as parenteral and oral means. In the first place, it enables gastrointestinal metabolism and the hepatic first-pass effect to be avoided; in addition, it is a non-invasive method which allows plasma levels of active principle to be maintained constant for long periods of time, comparable to those following an intravenous infusion. The use of patches for transdermal administration of active principles, however, is considerably limited by the inevitable presence of a latent period prior to the onset of the pharmacological effect. This latent period before the appearance of the therapeutic action can even reach 10 hours. This inconvenient latent period, prior to substance transportation through the skin, is related to patch and skin characteristics. All commercially available patches have this shortcoming.

The current inventors have recently developed a substance delivery device for dermal and transdermal use which is innovative compared to the aforesaid traditional devices. Said device, having been described in patent WO02/030402 by the same applicants, consists of a single layer of material in which are included all the elements that constructionally characterise a transdermal patch, that is to say active principle, adhesive and film-forming or structuring agent, that provides the mechanical structure.

The device appears as a resistant film and, being a single layer containing active principle/adhesive/structuring agent, has the same composition on both sides. Moreover the device in question is soluble in water, it is not adhesive as such but becomes adhesive by interacting with water which wets the skin or the device itself.

In recent studies, the current inventors have found that the single layer of WO02/030402 enables to achieve a transport of active principle through the skin which is very rapid and shows much shorter lag times compared to those of traditional patches.

However, said device still presents a series of drawbacks which substantially limit its use for transdermal administration of active principles.

Firstly, in the case of transdermal application, it is desirable that active principle transport be maintained constant for long periods of time i.e. more than 24 hours. Instead, with the device described in WO02/030402, it is observed that, after applying to the skin, the transport of active principle through the skin slows down progressively and stops after a period of about 24 hours.

Moreover, a further limitation to using said device for transdermal drug administration is the absence of protection on the outer surface i.e. that not in contact with the skin. In this respect, when used with drugs for which transdermal administration is intended, the possibility that one side of the layer could disperse active principle into the environment or onto any clothes with which it comes into contact may not be acceptable. It is therefore necessary to find a means of insulating said exposed surface without modifying the application method of the is device, achieved by using water to wet the skin in the area of application.

Therefore, in order to present the transdermal use of said bioadhesive film, a solution to the aforesaid drawbacks must be found.

### SUMMARY OF THE INVENTION

The current inventors have now discovered that the aforesaid problems encountered with the device described in WO02/030402 can be solved by covering one of the two surfaces of said device with a protective layer able to limit water evaporation. The device obtained in this manner, when applied to the skin by the procedures already described, is capable of producing active principle transport kinetics which are completely original and especially favourable when compared to those of both traditional transdermal patches and of the same device with no protection.

### BRIEF DESCRIPTION OF THE DRAWINGS

FIG. 1 shows the permeation profile of lidocaine from the patch obtained with the procedure described in example 1a or from a patch in accordance with WO03/020402, having composition identical to that of the active layer of the patch in example 1a but with no insulating layer.

FIG. 2 shows the permeation profile of diclofenac from the patch obtained with the procedure described in example 2a, from a commercially available patch (Flector®, Bayer) and from a patch in accordance with WO03/020402, having composition identical to that of the active layer of the patch in example 2a but with no insulating layer.

FIG. 3 shows a permeation profile of estradiol from the patch obtained with the procedure described in example 3a and from a commercially available patch (Estraderm MX®, Novartis Farma).

### DETAILED DESCRIPTION OF THE PREFERRED EMBODIMENT

The present disclosure concerns a two-layered patch for the dermal or transdermal administration of one or more active principles consisting of a first layer having a homogeneous composition and comprising at least an active principle, a water-soluble film-forming agent and a hydrophilic adhesive polymer, and of a second layer joined in a permanent manner to the first and having a water vapour permeability of less than 500 g/m² in 24 hours.

In particular, in the patch of the present disclosure, the active principle/adhesive/film-forming polymer mixture, which constitutes the first layer, is spread onto a film of material which has a solely insulating function, and is then dried until the surface exposed to air loses its adhesiveness.

Therefore, the patch of the invention consists of a first layer containing the active principle, known as the active layer, which is not self adhesive, as is the case with normal transdermal patches, but acquires adhesiveness only after contact with water, and of a second layer, known as the insulating layer, which restricts water evaporation from the active layer after the latter is applied to the skin.

The active layer has a thickness preferably between 10 and 1000 μm. The term ‘active principle’ according to the present disclosure means any substance with pharmacological or cosmetic activity. However, in accordance with a particularly preferred embodiment of the patch of the present disclosure, the active principle is preferably a drug.

The film-forming agent and the adhesive polymer can be present in the active layer as two distinct components or, in the case of polymers which possess both properties, as a single component.

Generally, the hydrophilic adhesive is chosen from the group consisting of polyvinylpyrrolidone preferably of molecular weight between 2,000 and 1,500,000, tragacanth gum, gum arabic, karaya gum, xanthan gum, pectin and polyaminomethacrylate adhesives.

According to a preferred embodiment of the present disclosure the adhesive is selected from the group consisting of polyvinylpyrrolidone, polyaminomethacrylate and mixtures thereof. Of the polyaminomethacrylate adhesives, particularly preferred is the butylmethacrylate/(2-dimethylaminoethyl)-methacrylate/methylmethacrylate copolymer in which the ratio between the monomers is 1:2:1, and is commercially known by the name Eudragit E100.

The term ‘film-forming agent’ according to the present disclosure means an agent capable of forming a film after being dried.

The water soluble film-forming agent used in the patch of the present disclosure is generally chosen from the group consisting of carboxymethylcellulose, chitosan, aqueous dispersions of acrylic and methacrylic polymers and polyvinyl alcohol. According to a more preferred embodiment said film forming agent is polyvinylalcohol and more preferably said polyvinylalcohol is characterized by having a molecular weight between 500 and 115,000 and degree of hydrolysis between 86 and 99%.

The insulating layer consists preferably of a film of polyethylene, polyolefin, ethylene vinyl acetate, polyurethane, polyester, polypropylene, polyvinyl chloride, aluminium, fabric or cross-linked polyvinyl alcohol. Preferably said film has a water permeability less than 250 g/m² in 24 hours.

The patch of the invention can also include at least one substance acting as absorption promoter and/or humectant and/or plasticiser. Preferably said substance is chosen from the group consisting of glycerine, ethyl alcohol, propylene glycol, polyethylene glycol of molecular weight from 400 to 6000, sorbitol, phospholipids, terpenes, soya lecithin, phosphatidylcholine, cholesterol, cyclodextrin, isopropyl myristate, oleic acid, polysorbate 80 and diethylene glycol monoethyl ether (commercially known by the name Transcutol®, Gattefossee, France).

The active layer of the patch of the present disclosure also contains water in a maximum quantity of 20%, preferably of from 1 to 15%.

Upon use, the non-insulated surface of the patch of the present disclosure is applied to the skin with water, for example by wetting the skin before applying the patch or by briefly immersing the patch in water, whereas the other surface of the patch remains insulated from the exterior by means of an insulating layer, which limits water evaporation. The patch applied to the skin in this manner remains compact and attaches firmly to the skin. The patch of the present disclosure precisely covers all the skin surface of application and adapts perfectly to skin wrinkles and folds, considerably increasing surface area of contact and thus transport of active principle into the skin.

As will be illustrated in greater detail by the experimental examples to follow, due to the effect of the composition of the active principle/adhesive/filmogen mixture and the presence of the insulating layer, the patch of the present disclosure gives rise to kinetics of substance transport through the skin which are particularly favourable when compared with those typical of both normal self-adhesive transdermal patches and of the single layer described in WO02/030402. In particular, unlike that observed with traditional patches, initial transport of a substance through the skin is very rapid and the lag time is practically eliminated. In addition, the duration of active principle transport is considerably greater than that observed with the single layer of WO02/030402 which does not possess an insulating layer. Finally, with the two-layered patch of the present disclosure, the amount of active principle transported through the skin is considerably greater, for the same duration of application, than that transported in the case of either traditional self-adhesive patches or of the single layer of WO02/030402.

The patch of the present disclosure is useful for the dermal or transdermal administration of any active principle, whether hydrophilic or lipophilic in nature. However, in view of its behaviour in terms of kinetics of active principle transport through the skin, the patch of the present disclosure is particularly suitable for the transdermal administration of active principles.

Numerous drugs can produce a rapid effect, benefiting from this original transport behaviour, for example the analgesics fentanyl, butorphanol, morphine, buprenorphine, naloxone, codeine; local anaesthetics such as lidocaine, anti-acne drugs like retinoic acid; anti-angina drugs like nitroglycerin, isosorbide dinitrate, nifedipine, nicardipine; antiarrhythmics like timolol; antibacterials like amikacin, cephalosporins, macrolides, tetracyclines, quinolones, nitrofurantoin; anti-convulsives like carbamazepine, phenobarbital, nitrazepam; antidepressants like tricyclics, bupropion, sertraline, pergolide, fluoxetine; anti-rheumatics like diclofenac, ibuprofen, piroxicam, ketoprofen, thiocolchicoside, methotrexate; sex hormones like progesterone, testosterone, estradiol, levonorgestrel; anti-fungals like clotrimazole, ketoconazole, miconazole; anti-hypertensives like sotalol, alprenolol, captopril, enalapril, felodipine, nicardipine, reserpine; anti-hypothyroid drugs like thyroxine; anti-malarials like artemesine, cinchonidine, primaquine; anti-migraine drugs like ergotamine, sumatriptan, rizatriptan; anti-nausea drugs like domperidone, chlorpromazine, methoclopramide, scopolamine, tetrahydrocannabinoids; skin lighteners like hydroquinone, hydroquinine; dopamine receptor antagonists like pergolide, bromocriptine; muscle relaxants like thiocolchicoside, diazepam; sclerosing agents like ethanolamine, sodium ricinoleate; vitamins like A, B, C, E and precursors or various agents like oxybutynin, finasteride, erythropoetine.

A further aspect of the present disclosure is a process for preparing the patch of the present disclosure comprising the following steps:

a) a mixture is prepared, in water, comprising the hydrophilic adhesive, preferably in an amount between 1% and 50% w/w, the film-forming agent, preferably in an amount between 1% and 60% w/w, the active principle, preferably in an amount 0.1% and 20% w/w and, optionally, one or more substances with the properties of absorption promoters/humectants/plasticisers, preferably in an amount between 0.5% and 20%. The water content in said mixture is preferably between 50% and 85%.
b) the mixture obtained in step a) is spread in a thin layer, preferably between 30 μm and 3000 μm in thickness, onto an insulating film;
c) the patch is dried until a residual moisture content of less than 20% is achieved.

Preferably the preparation of the mixture in step a) is carried out by the following steps:

a1) an aqueous solution of the film-forming agent is prepared;
a2) the hydrophilic adhesive polymer and the active principle are added to the solution obtained in step a1 in the form of an aqueous solution, micronized particles or emulsion.

The following non-limiting examples serve to further illustrate the present disclosure.

### Example 1

### 1a) Preparation of a Lidocaine Hydrochloride-Containing Patch Non-Adhesive in the Dry State

A mixture having the following composition is prepared:

| | |
|---|---|
| lidocaine hydrochloride | 2.00 g |
| PVA 83400, degree of hydrolysis 87.5% | 12.4 g |
| lauric acid | 2.48 g |
| adipic acid | 0.49 g |
| Eudragit E100 | 4.29 g |
| glycerine | 0.27 g |
| sorbitol | 2.8 g |
| water | remainder to 100 g |

In detail, the PVA is hydrated in 49 ml water for 12 hours. It is then gradually heated to 90° C. and stirred until completely dissolved. Separately, the adhesive is prepared by adding Eudragit E100, lauric acid and adipic acid to 21.27 ml of water, previously heated to a temperature of 78-82° C. The mixture is stirred for about 30 minutes, maintaining the temperature constant. The mixture is subsequently cooled to 60° C. and 0.27 g of glycerine are added. In another container the lidocaine hydrochloride is dissolved in 5 ml of water. The adhesive solution, the lidocaine solution and the sorbitol are added to the PVA solution in that order.

The mass obtained is spread as a thin film (300 μm thickness) using a doctor blade device, such as that supplied by BYK-Gardner (Silver Spring, USA), onto an occlusive protective layer (Scotchpak 1220, 3M, USA). The entirety is placed in a ventilated oven for 30 minutes at a temperature of 80° C. The patch obtained is one layer of 50 μm thickness containing a quantity of lidocaine equal to 0.4 mg/cm².

### 1b) In Vitro Permeation Experiments through Rabbit Ear Skin

The in vitro permeation of a drug from patches prepared in example 1a) was analysed using a vertical Franz diffusion cell and a barrier consisting of rabbit ear skin, in accordance with the protocol described by Cristina Padula et al, in Journal Controlled Rel 88, 277-285, 2003.

Patches without an insulating layer and consisting only of the active layer of the patches prepared in example 1a), prepared as described in WO02/030402, were used as controls.

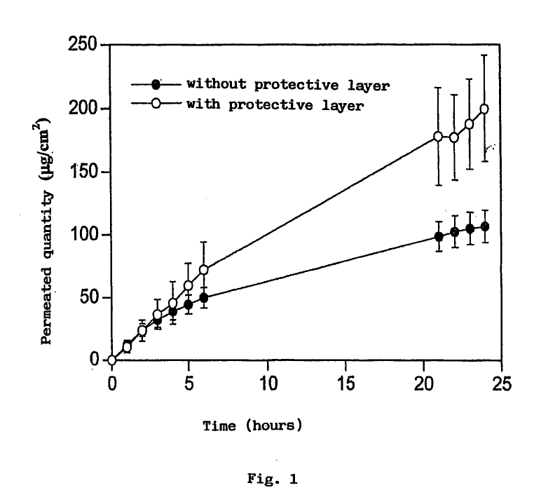

FIG. 1 shows the average quantity of lidocaine permeated with time per cm² of patch for each of the two types of patches tested.

The data obtained indicate that both patches tested give rise to a very rapid active principle transport through the skin, with no lag time.

However, in the case of the patch of the present disclosure, that is to say in the presence of the protective layer, the total amount of lidocaine permeated as well as the duration of drug transport through the skin are considerably greater than with the single layer patch.

### Example 2

### 2a) Preparation of a Diclofenac Potassium-Containing Patch Non-Adhesive in the Dry State

A mixture is prepared having the following composition:

| | |
|---|---|
| diclofenac potassium | 4.72 g |
| PVA 83400, degree of hydrolysis 87.5% | 1.13 g |
| PVP K 90 | 14.47 g |
| PEG 400 | 8.14 g |
| Lutrol F 127 | 3.85 g |
| Eugenol | 3.39 g |
| Menthol | 2.71 g |
| water | remainder to 100 g |

In detail, the PVA is hydrated in 4.52 ml of water for 12 hours. It is then gradually heated to 90° C. and stirred until completely dissolved. Separately, a solution of Lutrol F127 (Basf, Germany) is prepared, dispersing the triblock copolymer in 12.22 ml of water, leaving under agitation at ambient temperature for about one hour then maintaining the solution obtained at about 4° C. for at least 2 hours. 1.29 g of the drug are added at ambient temperature while stirring. Separately, the adhesive is prepared by slowly adding the PVP K 90 (Basf, Germany) to a solution of PEG 400 in 44.85 ml of water, and stirring gently for 12 hours to favour polymer hydration. The components are mixed together, the remaining quantity of drug is added and, once dissolved, the eugenol and the menthol are added to the mass. The mixture is slowly stirred for one hour at 60° C., to favour mixing of the components. The mass obtained is spread in the form of a thin film (600 μm thickness) using a doctor blade device, such as that supplied by BYK Gardner (Silver Spring, USA) onto an occlusive backing sheet (Cotran 9702, 3M, USA). The patch obtained is one layer of 40 μm thickness containing a quantity of diclofenac equal to 2.5 mg/cm².

2b) In Vitro Permeation Experiments through Rabbit Ear Skin

The in vitro permeation of a drug from patches prepared in example 2a) was analysed using a vertical Franz diffusion cell and a barrier consisting of rabbit ear skin (Cristina Padula et al, in Journal Controlled Rel 88, 277-285, 2003). Commercially available patches containing 1 mg/cm² diclofenac (Flector®, Bayer) and patches consisting of only the active layer of the patches prepared in example 2, prepared as described in WO 02/030402, and without insulating layer, were used as controls.

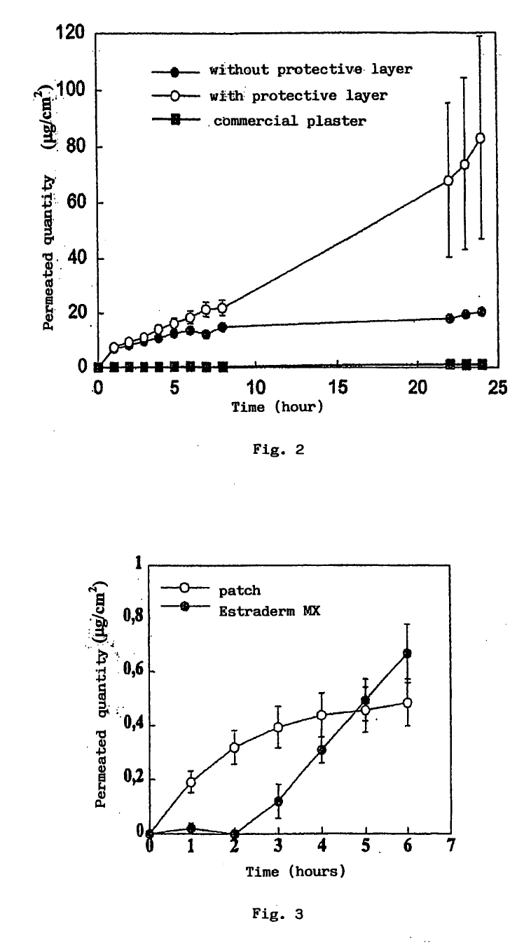

FIG. 2 shows the average quantity of diclofenac permeated with time per cm² of patch for each of the three types of patches tested.

The results obtained demonstrate that the patch of the present disclosure has favourable characteristics compared to both commercially available patches containing the same active principle and the patches described in WO02/030402. In fact, compared to commercially available patches where a lag time of some hours and very low amounts of permeated drug are observed, the patches of the present disclosure provide a very rapid initial transport through the skin with no lag time and greater amounts of transported drug, this being a characteristic also observed with the patch of WO02/030402.

Furthermore, compared to the patches of WO02/030402, the patches of the present disclosure have the further advantage of providing a transport duration that is considerably extended as well as a greater quantity of transported drug. In particular, while, in the case of the WO02/030402 patches drug transport slows down gradually over time and stops after 8 hours, with the patches of the present disclosure such transport is sustained for over 24 hours.

### Example 3

### a) Preparation of an Estradiol-Containing Patch Non-Adhesive in the Dry State

A mixture is prepared having the following composition:

| | |
|---|---|
| estradiol | 0.08 g |
| PVA 83400, degree of hydrolysis 87.5% | 12.40 g |
| lauric acid | 2.48 g |
| adipic acid | 0.49 g |
| Eudragit E100 | 4.29 g |
| glycerine | 7.17 g |
| β-cyclodextrin | 0.40 g |
| water | remainder to 100 g |

In detail, the PVA is hydrated for 12 hours in 49 ml of water. It is then gradually heated to 90° C. and stirred until completely dissolved. Separately, the adhesive is prepared by adding Eudragit E 100, lauric acid and adipic acid to 19.47 ml of water, previously heated to a temperature of 78-82° C. The mixture is stirred for about 30 minutes, maintaining the temperature constant. The mixture is subsequently cooled to 60° C. and 7.17 g of glycerine are added. In another container the estradiol and cyclodextrin are dissolved in 4.22 ml of water in another container. The adhesive solution and the estradiol solution are added to the PVA solution in that order.

The mass obtained is spread in the form of a thin film (400 μm thickness) using a doctor blade, such as that supplied by BYK-Gardner (Silver Spring, USA), onto an occlusive backing sheet (Scotchpak 1220, 3M, USA).

The patch obtained is one layer of 60 μm thickness containing a quantity of lidocaine equal to 7.5 μg/cm².

### b) In Vitro Permeation Experiments through Rabbit Ear Skin

The in vitro permeation of a drug from patches prepared in example 3a) was analysed using a vertical Franz diffusion cell and a barrier consisting of rabbit ear skin (Cristina Padula et al, in Journal Controlled Rel 88, 277-285, 2003). Commercially available patches each containing 68.2 μg/cm² of estradiol (Estraderm MX®) were used as a control.

FIG. 3 shows the average quantity of estradiol permeated with time per cm² of patch for each of the two types of patch tested. The results obtained demonstrate that the patch of the present disclosure, contrary to commercial patch, provides an immediate drug release with no lag time.

What is claimed is:

1. Two-layered patch for dermal or transdermal administration of active principles comprising:
a) a first layer having a homogeneous composition and comprising at least one active principle, a water-soluble film-forming agent selected from the group consisting of carboxymethylcellulose, chitosan, aqueous dispersions of acrylic and methacrylic polymers and polyvinyl alcohol, and a hydrophilic adhesive polymer, chosen from the group consisting of polyvinylpyrrolidone, tragacanth gum, arabic gum, karaya gum, xanthan gum, pectin and polyaminomethacrylate adhesives, said first layer having a residual humidity lower than 20%; and
b) a second layer joined in a permanent manner to the first and having a permeability to water vapour of less than 500 g/m² in 24 hours,
provided that when said adhesive is polyaminomethacrylate and it is butylmethacrylate/(2-dimethylaminoethyl)-methacrylate/methylmethacrylate copolymer in which the ratio between the monomers is 1:2:1, said water-soluble film-forming agent must be different from carboxymethylcellulose.

2. Patch as claimed in claim 1 wherein said residual humidity is comprised between 1 and 15%.

3. Patch as claimed in claim 1 wherein said adhesive is selected from the group consisting of polyvinylpyrrolidone.

4. Patch as claimed in claim 1, having average molecular weight between 2,000 and 1,500,000, polyaminomethacrylate and mixtures thereof.

5. Patch as claimed in claim 1 wherein said polyaminomethacrylate butylmethacrylate/(2-dimethylaminoethyl)-methacrylate/methylmethacrylate copolymer in which the ratio between the monomers is 1:2:1.

6. Patch as claimed in claim 1, wherein said film forming agent is polyvinylalcohol.

7. Patch as claimed in claim 1, wherein said polyvinylalcohol has a molecular weight between 500 and 115,000 and degree of hydrolysis between 86 and 99%.

8. Patch as claimed in claim 1 wherein said second layer (b) comprises at least one selected from the group consisting of: polyethylene, polyolefin, ethylene vinyl acetate, polyurethane, polyester, polypropylene, polyvinyl chloride, cross-linked polyvinyl alcohol, aluminium and fabrics.

9. Patch as claimed in claim 1 further comprising at least one substance acting as absorption promoter and/or humectant and/or plasticiser.

10. Patch as claimed in claim 9 wherein said substance is at least one selected from the group consisting of: glycerine, ethyl alcohol, propylene glycol, polyethylene glycol of molecular weight between 400 and 6000, sorbitol, phospholipids, terpenes, soya lecithin, phosphatidylcholine, cholesterol, cyclodextrin, isopropyl myristate, oleic acid, polysorbate 80 and diethylene glycol monoethyl ether.

11. Patch as claimed in claim 1 wherein the first layer has a thickness of between 10 and 1000 μm.

12. Process for preparing a patch in accordance with claim 12 comprising the following steps:
a) a mixture is prepared, in water, comprising the hydrophilic adhesive, the film-forming agent, the active principle, and, optionally, one or more substances with the properties of absorption promoters/humectants/plasticisers;
b) the mixture obtained in step a) is spread in a thin layer onto an insulating film; and
c) the patch is dried until a residual humidity of less than 20% is achieved.

13. Process as claimed in claim 12, wherein the mixture prepared in step a) comprises between 1% and 50% w/w of hydrophilic adhesive, between 1% and 60% w/w of film-forming agent, between 0.1% and 20% w/w of active principle, between 50% and 85% w/w of water, and, optionally, between 0.5% and 20% w/w of one or more substances with the properties of absorption promoters/humectants/plasticisers.

14. Process as claimed in claim 12 wherein the mixture of step a) is prepared by a process comprising the following steps:
a1) an aqueous solution of the film-forming agent is prepared; and
a2) a solution of hydrophilic adhesive polymer and the active principle in the form of an aqueous solution, micronized particles or emulsion is added to the solution obtained in step a1).

15. Process as claimed in claim 12 wherein in step b) said mixture is spread to give a layer of thickness between 30 and 3000 μm.
